(19) United States
(12) Patent Application Publication
(10) Pub. No.: US 2008/0228191 A1
(43) Pub. Date: Sep. 18, 2008

(54) Femoral elevator

(75) Inventors: Emily Downs, Grand Rapids, MI (US); Adam Bastian, Chester, NY (US); Nicholas Jon Lavigna, Mountain Top, PA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/717,578

(22) Filed: Mar. 13, 2007

(51) Int. Cl. A61B 17/56; A61B 17/00

(52) U.S. Cl. 606/90; 606/100; 606/207

(57) ABSTRACT

A bone retractor that may be used as a femoral elevator. The bone retractor includes a clamp for attaching the bone retractor to an operating room bed rail, a post attached to the clamp via a hinged joint, a mount slidably attached to the post and a hook coupled to the mount which in turn is attached to the post. A shaft passes through the interior of the post and attaches to clamp. The clamp has a lower jaw and an upper jaw. An opening is formed in the lower jaw and an end of the shaft is engaged in the opening. The shaft has two sections with a universal joint connecting these two sections. The born retractor is attached to the operating room bed rail by applying clamp to the bed rail. With bone retractor attached to the bed rail, the post is lowered towards the wound. The hook is placed under the bone, and the bone exposed by holding the hook or the post and lifting the bone out of the wound. A knob is tightened to lock the post and consequently the hook in the raised position while the surgeon performs the surgery. When the surgeon is ready, the bone is lowered in the wound by loosening the knob and slowly placing the bone in the desired position in the wound.

### BACKGROUND OF THE INVENTION

1. Field of the Invention

The present invention relates to a system and method for surgical retraction. Particularly, this invention relates to a system and method for retracting a femur during hip surgery.

2. Description of Related Art

Hip and shoulder operations have been performed for decades. In these operations the surgeon makes an incision and exposes the joint. Then the bone is lifted out to allow the surgeon to visualize and resect the bone. In the past surgeons have used specialized orthopedic surgical operating tables, retractor systems and/or leg positioning by surgical staff to visualize and resect the femur. Orthopedic operating tables are expensive and utilize mechanical advantage such as gears to elevate the position of the femur. Often it is another member of the team that operates the controls of the operating table and therefore, the surgeon does not have control of the positioning of the leg. The lack of control by the surgeon and the use of mechanical advantage have been known to cause patient injury such as ankle fractures. Many available retractor systems are cumbersome, require more than one person to use, and also use mechanical advantage such as gears to lift bone. There is a need for a retractor system that is operable by one person, easy to use, inexpensive and that does not use mechanical advantage to lift the bone.

### SUMMARY

The present invention provides a bone retractor that overcomes the shortcomings of the prior art. The bone retractor of the present invention may be used as a femoral elevator. The femoral elevator includes a clamp for attaching the femoral elevator to an operating room bed rail, a post attached to the clamp via a hinged joint, a mount slidably attached to the post and a hook coupled to the mount which, in turn, is attached to the post. A shaft passes through the interior of the post and attaches to the clamp via a translation piece. The clamp has a lower jaw and an upper jaw. An opening is formed in the lower jaw and an end of the translation piece is engaged in the opening. The shaft has two sections with a universal joint connecting these two sections. A handle is attached to one end of the shaft, and turning the handle brings the lower and upper jaw together.

In use the femoral elevator is attached to the operating room bed rail by applying clamp to the bed rail. The upper jaw and lower jaw are placed around the bed rail and the handle is turned. The turning of handle draws the lower jaw towards the upper jaw thereby making claw and claw clamp tight around the bed rail. With femoral elevator attached to the bed rail, the hook is placed under the bone and attached to the mount. The bone is exposed by holding the hook or the post and lifting the bone out of the wound. A knob is tightened to lock the post and consequently the hook in the raised position while the surgeon performs the surgery. When the surgeon is ready, the bone is lowered in the wound by loosening the knob and slowly placing the bone in the desired position in the wound.

### BRIEF DESCRIPTION OF THE DRAWINGS

FIG. 1 shows an isometric view of one embodiment of the bone retractor of the present invention.

FIG. 2 shows an isometric view of a lower jaw of a clamp used in the bone retractor of FIG. 1.

FIG. 3 is an isometric view of a shaft used in the bone retractor of FIG. 1.

FIG. 4 shows an isometric view of an upper jaw of a clamp used in the bone retractor of FIG. 1.

FIG. 4A shows a bottom view of the upper jaw of FIG. 4.

FIG. 5 is an isometric assembly drawing showing an assembly of a post and a mount used in the bone retractor of FIG. 1.

FIG. 6 is also an isometric assembly drawing showing an assembly of a post and a mount used in the bone retractor of FIG. 1.

FIG. 7 shows an isometric view of a housing assembly that forms part of the mount seen in FIG. 5.

FIG. 8 shows isometric view of a hook used in the bone retractor of FIG. 1.

FIG. 9 shows a translation piece that is connected to the shaft of FIG. 3, the upper jaw of FIG. 4 and with the lower jaw of FIG. 2.

FIG. 10 shows an isometric view of connector piece used in bone retractor in FIG. 1.

FIG. 11 shows a cross sectional view of an embodiment of a bone retractor.

FIG. 11A is an enlarged cross sectional view of the ratchet connection shown in FIG. 11.

### DETAILED DESCRIPTION

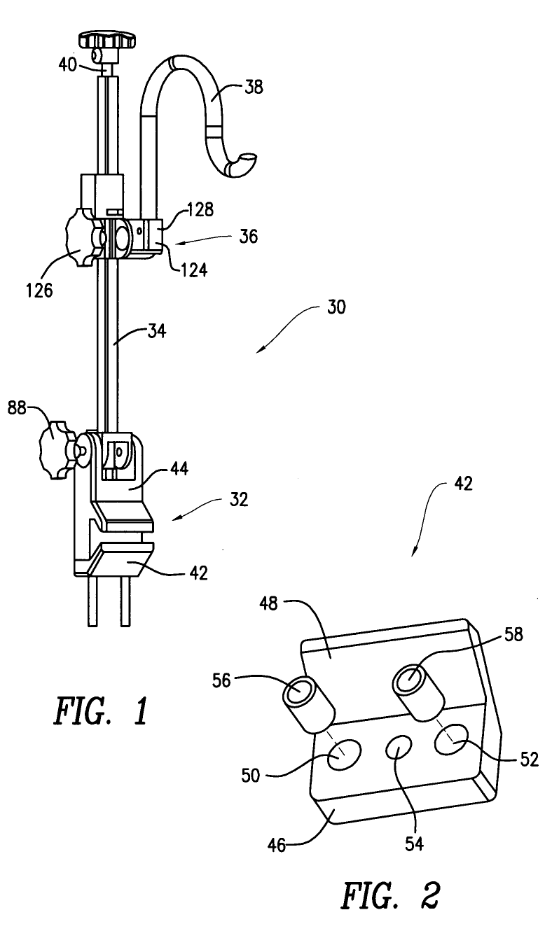

FIG. 1 shows a bone retractor suitable for use as a femoral elevator. Femoral elevator 30 includes a clamp 32 for attaching the femoral elevator to an operating room bed rail. A hollow post 34 is attached to clamp 32 via a hinged joint. A mount 36 is slidably attached to post 34. A hook 38 is coupled to mount 36 which in turn is attached to post 34. A shaft 40 passes through the interior of hollow post 34 and engages with translation piece 35 (FIG. 9) which attaches to clamp 32.

Clamp 32 has a lower jaw 42 and an upper jaw 44. FIG. 2 shows an isometric view of lower jaw 42. Lower jaw 42 has a base 46 and a claw 48. Base 46 has three openings 50, 52 and 54. Openings 50 and 52 are sized to accept sleeve bearings 56 and 58 respectively. Opening 54 may be a through hole, and the end 55 of translation piece 35 may pass through it and a cap may be placed on the free end of translation piece.

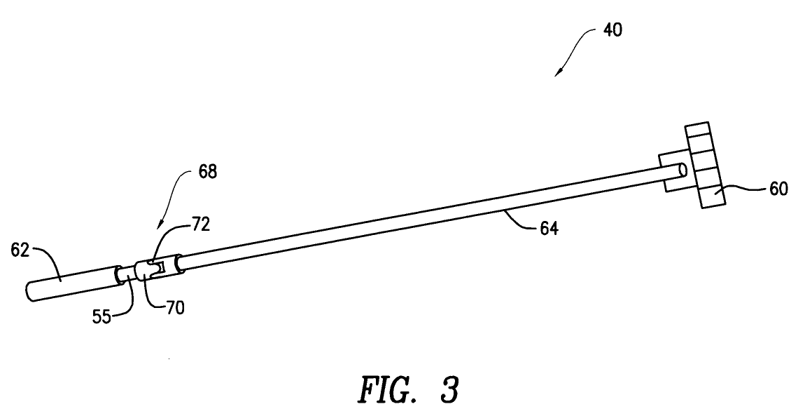

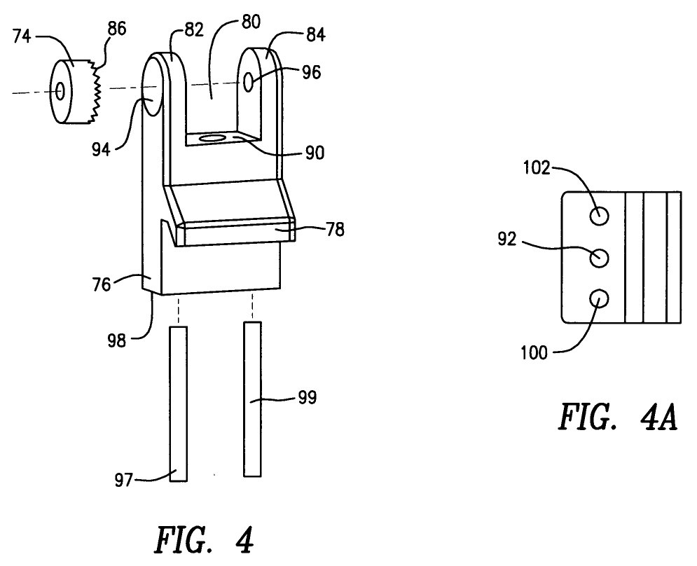

FIG. 3 shows shaft 40 with a handle 60 attached to the end opposite to the end that engages with the translation piece. A cylindrical piece 62 may have an internal hex feature and may be attached to the end of the shaft opposing the end with handle 60. Cylindrical piece 62 may be engaged with end 63 and body portion 65 of translation piece 35 and be assembled with lower jaw 42. End 63 of translation Piece 35 may have an external hex feature that engages with the internal hex feature of cylindrical piece 62. A portion of translation piece 35 may have threads 67 that engage with hole 92 (FIG. 4A) of the upper jaw 42. Translation piece 35 has an end 55 opposite the external hex feature of translation piece 35. End 55 may be a smooth shaft that passes through hole 54 of the lower jaw 42. Long portion 64 and cylindrical piece 62 are connected to each other via a universal joint 68 that allows rotational motion between long portion 64 and cylindrical piece 62. Universal joint 68 may be of one of many different designs known to one skilled in art. For example, universal joint 68 may include pivot pins 70 and 72 that allow rotational motion between long portion 64 and cylindrical piece 62 around two axes. Shaft 40 is housed in post 34 and passes through an opening in upper jaw 44.

FIG. 4 shows an isometric view of upper jaw 44. FIG. 4A shows a bottom view of upper jaw 44 of FIG. 4. Upper jaw 44 has a bushing 74, a base 76 and a claw 78 projecting from base 76. One end of base 76 is machined to form a notch 80 with cantilever beams 82 and 84 forming two sides of notch 80. Cantilever beam 82 has bushing 74 inserted in its free end. Bushing 74 has teeth 86 formed on one face. A knob 88 (FIG. 1) is connected to cantilever beam 82 such that when knob 88 is turned, teeth 86 engage with teeth on post 34 to lock post 34 in its position. Notch 80 has a base 90 with a hole 92 formed in base 90. The portion of hole 92 on the opposite end of the cantilever beams 82 and 84 may be threaded. Holes 94 and 96 are formed in cantilever beams 82 and 84 respectively. Upper jaw 44 has a bottom surface 98 at the end opposing cantilever beams 82 and 84. Two holes 100 and 102 are formed in bottom surface 98. Dowel rods 97 and 99 are press fit in holes 100 and 102 respectively and are slidably engaged to sleeve bearings 58 and 56 (FIG. 2) respectively. Claw 78 cooperates with claw 48 to attach femoral elevator 30 to the operating room bed rail.

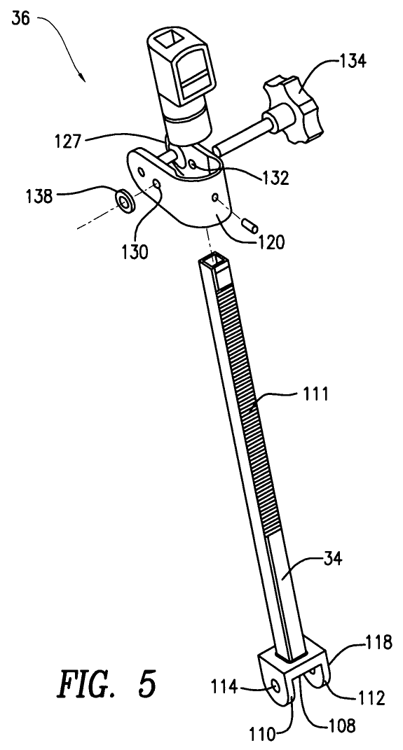

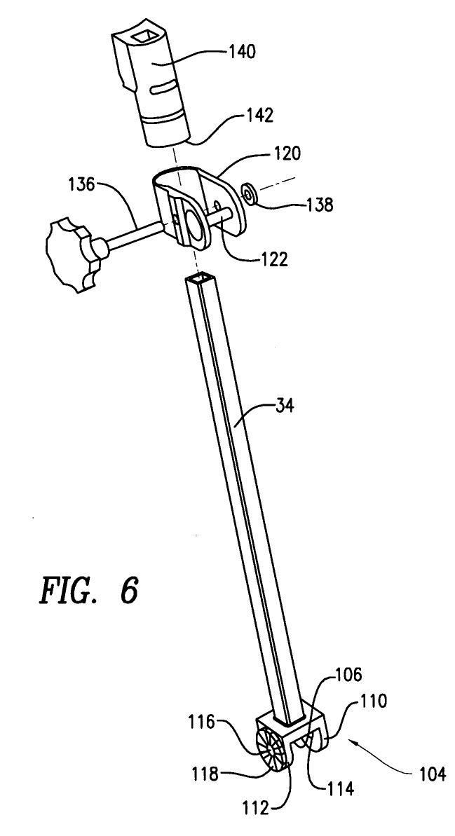

FIGS. 5 and 6 show post 34. Post 34 has a forked end 104 having a notch 106. Notch 106 has a base 108 with a hole (not shown) formed in base 108 and cantilever beams 110 and 112 forming two sides of notch 106. Holes 114 and 116 are formed in cantilever beams 110 and 112 respectively. Teeth 118 are formed on the external surface of cantilever beam 112. When post 34 is assembled with clamp 32, teeth 118 face teeth 86 on bushing 74. When knob 88 is turned, teeth 86 engage with teeth 118 to lock post 34 in its position. Post 34 may have a rectilinear shape with a face of the external surface having a ratchet structure 111, such as teeth of appropriate shape.

FIGS. 5 and 6 also show mount 36 having a U-shaped clip 120. The free ends of U-shaped clip 120 have holes and a pin 122 passing through the holes. Pin 122 is fixedly attached to one end of U-shaped clip 120 while the other end of U-shaped clip 120 is free and can slide along the length of pin 122. Next to the holes with pin 122 is straight hole 130 and threaded hole 132 which are co-axial. A knob 134 having a threaded shaft 136 is inserted through holes 130 and 132 and a washer 138 is fixed on the end of shaft 136 opposite the knob. Turning knob 134 threads shaft 136 in threaded hole 132 resulting in ends of U-shaped clip 120 moving towards or away from each other. A connecting piece 124 (FIG. 10) having a passage 126 and a pocket 128 is mounted in U-shaped clip 120. Cylindrical passage 126 is at right angle to pocket 128. Pocket 128 holds hook 38. Passage 126 is passed over pin 122 thereby forming a hinged connection between connecting piece 124 and mount 36.

Connecting piece 124 contains holes 125 that are in a radial pattern around the end of cylindrical passage 126. The inner surface of the free end of the U-shaped clip has a radial pattern of rounded protrusions 127. Rounded Protrusions 127 mate with holes 125 on connector piece 124. When knob 134 is operated, the free ends of 120 move towards each other, forcing the rounded protrusions 127 to engage with the holes 125 and secure the orientation of the connector piece 124. The radial pattern of the rounded protrusions 127 and holes 125 allows the connector piece to be secured in a variety of orientations.

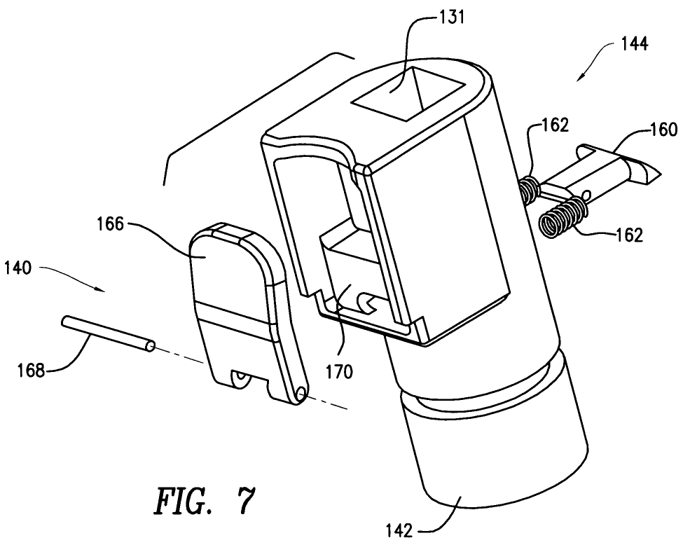

A housing assembly 140 is shown in FIG. 7. Housing assembly 140 has a circular end 142 and a rectangular hole 131 is formed along the longitudinal axis of housing assembly 140. Circular end 142 is sized to fit in the bottom of U-shaped clip 120. When Knob 134 of U-shaped clip 120 is tightened, circular end 142 is captured in U-shaped clip 120. Housing assembly 140 has a pawl like structure 144 that engages the teeth of ratchet structure 111. Housing 140 and post 34 have a pawl and ratchet connection between them and are therefore slidable with respect to one another. The pawl mechanism consists of a lifter piece 160, compression springs 162, release button 166 and pivot pin 168.

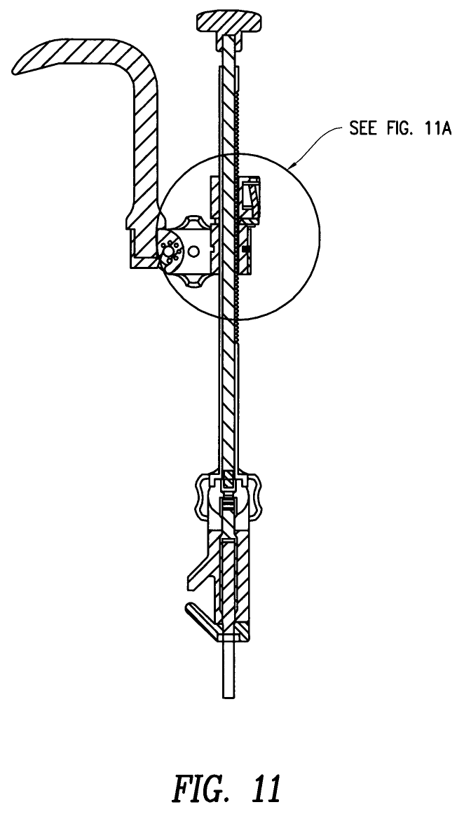

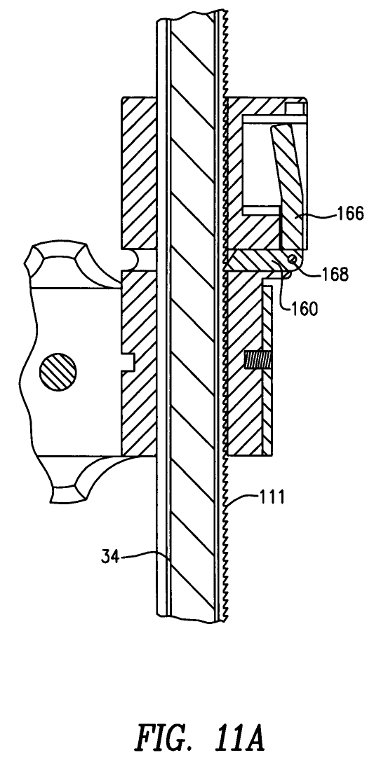

A cross sectional view of the ratchet connection is shown in FIG. 11. FIG. 11A is an enlarged view of the ratchet connection shown in FIG. 11. Lifter piece 160 engages with teeth of ratchet structure 111 when the release button 166 is in the neutral position and locks the mount 36 into position on the post 34. Compression springs 162 act to push the tongue of the lifter piece 160 into the ratchet teeth and then maintain that position even if an amount of force is applied in the opposite direction. The lifter piece 160 is designed so that the mount 36 will travel along ratchet structure 111 in the direction away from forked end 104 by applying force to the construct in that direction. The mount is moved in the direction toward forked end 104 by engaging release button 166, which is attached to the lifter piece 160 via the pivot pin 168. Engaging the release button 166 produces a pivot action with feature 170 of the housing 144, which pulls lifter piece 160 away from the ratchet surface 111 and compresses the springs 162. This action separates the tongue of lifter piece 160 from the ratchet teeth 111 and allows for movement of the housing along the shaft either towards or away from forked end 104.

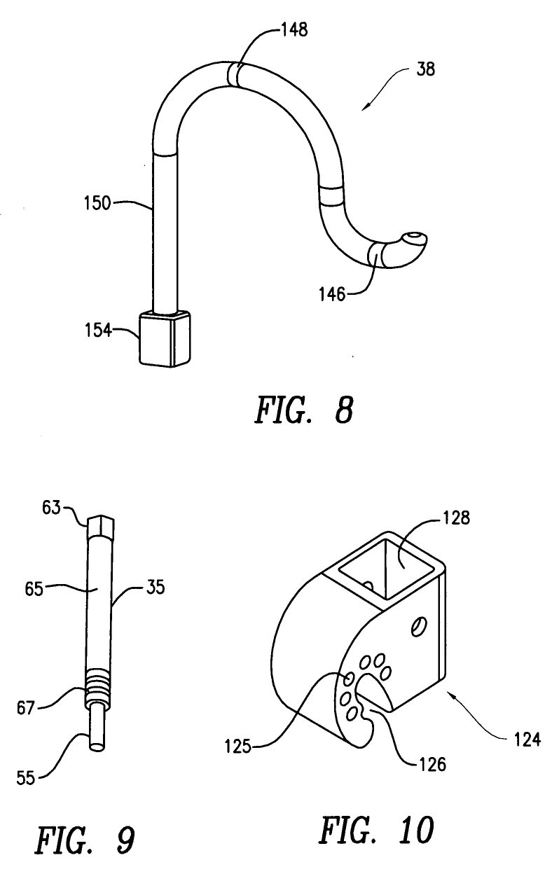

FIG. 8 shows an isometric view of hook 38. Hook 38 has a hook end 146 that is in shape similar to an arc of a circle. Hook end 146 is shaped so that it is suitable for placement under a bone. Hook end 146 transitions into a horizontal side 148. Hook 38 also has a vertical side 150 that connects with horizontal side 148 forming an approximate right angle between them. The free end of vertical side 150 is formed with an elongated portion 154 projecting there from. Elongated portion 154 is sized to have a snug fit in pocket 128 formed in connecting piece 124. The tip of hook 38 may be offset from the longitudinal axis of vertical side 150.

In use femoral elevator 30 is attached to the operating room bed rail by applying clamp 32 to the bed rail. Upper jaw 44 and lower jaw 42 are placed around the bed rail and handle 60 is turned. The turning of handle 60 draws lower jaw 42 towards upper jaw 44 thereby making claw 78 and claw 48 clamp tight around the bed rail. With femoral elevator 30 attached to the bed rail, knob 88 is loosened and post 34 lowered towards the wound. Mount 36 is slid along post 34 to place it in the vicinity of the wound. Hook 38 is placed under the bone, for example the femur that is being resected, and then elongated portion 154 of hook 38 is placed in cylindrical passage 128 formed in connecting piece 124. Alternatively, hook 38 is captured in connecting piece and then hook end 146 is placed under the bone. Knob 134 is tightened to securely capture hook 38 and connector piece 124 in mount 36. Next, the surgeon exposes the bone by holding the hook 38 or the post 34 and lifting the bone out of the wound. Knob 88 is tightened to lock post 34 and consequently hook 38 to hold the bone in the raised position while the surgeon performs the surgery. When the surgeon is ready, the bone is lowered in the wound by loosening knob 88 and slowly placing the bone in the desired position in the wound.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

We claim:

1. A surgical apparatus for manipulating bone within a surgical site, the apparatus comprising:
a clamp having a first jaw and a second jaw, the first jaw and the second jaw being adapted to attach to a surgical table;
a post pivotally attached to the second jaw;
a rod passing through the hollow post and attached to the first jaw, the rod adapted to move the first jaw and the second jaw closer; and
a hook slidably attached to the hollow post, wherein the hook can be placed under the bone within the surgical site and pivoting the hollow post moves hook in medial-lateral direction.

2. The surgical apparatus of claim 1, further comprising:
a mounting piece slidably attached to the hollow post, the mounting piece and the hollow post forming a pawl and ratchet connection such that the mounting piece can slide in one direction on the hollow post and not slide in the other direction unless a release is actuated.

3. The surgical apparatus of claim 2, further comprising:
opposing cantilever beams formed on the second jaw;
first set of teeth formed on at least one cantilever beam;
second set of teeth formed on one end of the hollow post; and
a first knob attached to the second jaw, the first knob when turned bringing the teeth formed on the cantilever beam in contact with teeth formed on the end of the post to lock the hollow post in its position.

4. The surgical apparatus of claim 3, wherein the hook is attached to the mounting piece.

5. The surgical apparatus of claim 4, wherein the rod further comprises;
a first section and a second section; and
a universal joint formed between the first section and the second section.

6. The surgical apparatus of claim 5, wherein the universal joint is located in a notch formed in the second jaw.

7. The surgical apparatus of claim 5, wherein the first or the second section of the rod is threaded.
